(19) United States
(12) Patent Application Publication
(10) Pub. No.: US 2006/0159778 A1
(43) Pub. Date: Jul. 20, 2006

(54) Method of treatment and prophylaxis of people's adiposity

(76) Inventor: Katie Seleznev, West Hartford, CT (US)

Correspondence Address: Katie Seleznev, 149 Brewster Road, West Hartford, CT, 06117, US

(21) Appl. No.: 11/039,402

(22) Filed: Jan. 19, 2005

(51) Int. Cl. A61K 36/76; A61K 36/18; A61K 31/717

(52) U.S. Cl. 424/725; 424/769; 514/57

(57) ABSTRACT

The present invention contains a non-medicamental method of the treatment and prophylaxis of an adiposity and also the normalization of a person's weight. This method is based on the inclusion in a diet during the treatment a big volume of the calorie-less, harmless, indigestible cellulose of plant or animal origin instead of a person's usually consumed food. The volume of this cellulose answers to the satisfaction of individual requirements of people in the food, which has calorific value covering only 20%-40% of the metabolism's daily power needs. Thanks to this, at all time during the treatment, the normal function of digestive organs is kept, the organism's stress reaction on the hunger sensation and the person's obesity syndrome hyperorexia are removed. And as a result, the volume of a consumed food decreases gradually and a person's weight reduces painlessly to the norm by utilizing the accumulated before excessive fat.

### BACKGROUND OF THE INVENTION

1. Technical Field

The present invention is the non-medicamental method in the decision of problem of the treatment and prophylaxis of an adiposity, and also the normalization of body weight. This method is based on the inclusion in the diet during treatment some food, that consists mainly of calorie-less and harmless cellulose of plant or animal origin.

2. Background Art

Now nearly one-third of adults in the USA are obese, and this phenomenon is spreading. On the average, American adults are on 25 pounds heavier than they were in the early 1960s. Uninterrupted growth of the adiposity among the population puts forward a problem of overweight people's treatment as one from principal tasks of the modern public health. It is known that the most frequent cause of the adiposity is the surplus consumption of the easy digestible food, in the first place carbohydrates with its rapid evacuation from the alimentary canal. This evokes a sensation of hunger, stimulates an appetite, the habit to a frequent and irregular reception of food, and the development of a carbohydrate dependence. Persons with obesity have the sensation of permanent hunger, the availability of an increased appetite and the syndrome hyperorexia (feeling empty, heightened appetite, and the bolting of a large volume of food), and that makes the struggle with this disease very difficult.

The employment of medicamental means and different fashionable methods for thinning down (starvation, an acceptance of diuretics, vomiting) entail some unfavorable side effects on organisms and on a metabolism, functioning of an alimentary canal and a vasculocardiac system. Moreover, thrown off with such difficulties, pounds come immutably back to their master. All that naturally restricts the struggle of people with their superfluous weight.

The most physiological in the treatment and prophylaxis of adiposity is the receiving of some food with the consideration of its calorific value. But widely advertised, this method, as the production of food-stuffs with instruction about a quantity of calories, doesn't give expected results in the struggle of lowering the number of persons with surplus weight and obesity.

Constant calculations of calories in some food with a consideration of daily energy needs, even for people with a normal weight, is a highly burdensome process, requiring a lot of psychological patience. Besides, the decrease of food volume and fat is accompanied by rapid emptying of a stomach and the permanent sensation of hunger, and owing to that people refuse to count food calories and volume. Yet a more complicated situation arises for people with a surplus of weight or with an acute obesity, suffering from the syndrome hyperorexia. The reduction of calorie quantity in some food and decreasing its volume raises by these people the sensation of hunger, and this causes their refusal from subsequent limitation in the food, does useless efforts in the psychological assistance to a sick person.

An aim at the present method is the fulfillment of the main practical task-normalization of obese people's weight by utilizing fat an excessively accumulative with them. At the same time, there are observing physiological principles of an organism's vital function, a removal of the hungry feeling during treatment by filling the stomach and bowels with a sufficient volume of some food, consisting mainly of calorie-less, inoffensive and indigestible cellulose of plant or animal origin. Rich in cellulose plant's parts, which didn't used for the food, are included in a person's ration.

### SUMMARY OF THE INVENTION

The present invention is the non-medicamental method for solving a problem—the normalization of people's weight, the treatment and prophylaxis of obesity. This method is based on the employment of diet, which consists mainly from the calorie-less cellulose of plant or animal origin. The indicated cellulose doesn't yield to the digest, slowly evacuates from an alimentary canal, and owing to this employs to fill the stomach and bowels in the sufficient volume for the satisfaction of individual requirements of people in some food. Thanks to that during the treatment, the syndrome hyperorexia by persons with obesity is removed.

Besides, to the basic calorie-less mass it is added a small quantity of the high-calorie food, which covers only a slight part of power requirements of the basic metabolism, but allows to keep up the normal function of digestive organs, and provides an organism with proteins, vitamins, and mineral salts. Other daily power requirements as people's basic energy metabolism (in calories and in condition of the rest), as physical loading becomes covered at the expense of utilization of the fat, which was with a surplus accumulated with these people. And as a result, this overweight reduces and individuals sense of hunger doesn't lose his or her physical activity. The main source of the cellulose of plant origin are barns, stems and leaves of wheat, rye, corn, beets, carrots, dills and other edible plants, the wood-pulp of birch, and fruit trees; and animal origin—fascicles and tendons of muscular tissues.

Stems and leaves of plants are used as in fresh, as in dried condition. Fresh stems and leaves are crushed into small pieces, and after boiling water added to salads and garnishes to different dishes. In dried condition, stems and leaves are grinded up to a powder, and then are used as an addition to bread, sausage, and other culinary products.

For the treatment of a person who has a weight in excess of 30%, a calorie-less mass makes 60%-65% of the whole volume of food; for a person who's weight is over the normal 20%-29%, a calorie-less mass makes 40%-60%; people who's overstep the weight on 19% and below the normal on the share of mass falls 20%-39%. The rest of the percents forms with some calorie food: meat and meaty products, milk, flour, vegetables, fruit, etc., with total calorie value, which covers 20%-40% of daily power requirements of the basal metabolism. And the volumetric part of the calorie food is basically composed of vegetables.

That way of receiving some food doesn't cause for people with obesity any problems after the treatment, in the time of the transfer on the usual ration with a quantity of calories, which cover completely the organism's power requirements.

### DETAILED DESCRIPTION OF THE PREFERRED EMBODIMENTS

The present invention includes the non-medicamental method in decision of one from practical medical and social tasks—the treatment and prophylaxis of the adiposity and also the normalization of a person's weight. A novelty of the present method is: it is based on the inclusion in a person's diet a big volume of one from substances that are indigestible in the stomach and bowels, harmless and unused now in food the cellulose of plant or animal origin. This gives a chance to decide some attendant practical problems, which make possible to do the treatment effective and cheap, quickly by terms of carrying out, easily endured by the patient, without any side effect on his or her organism.

The preference in a choice as medical means among natural and perhaps synthesized substances, which do not digest in the stomach and in bowels, is given for the present to cellulose of plant or animal origin, and this is due to:

a) the cellulose contains in usual food products, is harmless, doesn't digest in the stomach and bowels, leaving the person's organism in an unchanged condition;

b) one from main medical quality of cellulose is the mechanical filling of a stomach and bowels with a harmless, calorie-less, undigested mass, which evacuates slowly from an alimentary canal, ensuring a sensation of satiety, reduction of appetite, and consequently removes the syndrome hyperorexia by the person, which suffers from obesity; and as a result, he/she doesn't sense the feeling of hunger, and endures easy the expenditure of an excessively accumulative fat for the maintenance of an organism's active vital functions;

c) the cellulose increases volume of the food, but doesn't increase its calories. And irrepressible wants in eating of obese people are contented with the volume of calorie-less food.

d) the preference is given for the cellulose of plant origin, since it makes the greater part of cultivated plants, is the cheap and available source for its receiving, and owing to this the proposed method of the obesity treatment becomes generally available;

e) the employment for the cellulose in big quantities doesn't bring negative effects on the organism, and makes the physiological basis for a treatment;

f) an addition to some food different percent of the cellulose depending on the extent of exceeding a person's weight reduces the calories, regulates them, and ensures an individual approach to the volume of consumed food.

The main sources of cellulose of plant origin are barns, stalks, leaves of wheat, rye, corn, other edible plants, the wood of birch, fruit, and other trees. The cellulose of animal origin includes fascicles and tendons of muscular tissues. A plant's stalks and leaves are used as in fresh, as in dried condition. Fresh stems and leaves crush into small pieces, and after boiling add to salads and garnishes to different dishes. The stems and leaves in dried condition are grinded up to a powder, and then are used as an addition for a preparation of different bread, confectionery, sausage, and other culinary products. Instead of sugar are used its substitutes. The floury plant cellulose and crushed into powder cellulose of the animal origin are used as filling for sausage products, and culinary products from chopped meat. To impart its softness, consisting of a cellulose mass is exposed to cooking.

The treatment of people in excess of the weight on 30% and more than normal (people with obesity) the calorie-less mass makes 60%-65% of all food volume, for people with the weight more than normal of 20%-29% the calorie-less mass makes 40%-59%, and with exceeding of the body weight on 19% and lower the calorie-less mass makes 20%-39% of a food's total volume. Other percents of the volume fall to the calorie food: meaty, milky, and floury products, vegetables and fruits with total calorific value, which covers only 20%-40% of daily power requirements of the basal metabolism (in calories and in rest condition of an organism). This provides an organism, during the treatment, with proteins, carbohydrates, vitamins, mineral salts. The rest (60%-80%) of the power requirements on a person's basal metabolism and on the performance of physical loads is covered by an utilization of the excess accumulated by a person fat.

The acceptance of a low-calorie food, which contents a lot of the cellulose, keeps up the function of the alimentary canal, takes off the excruciating sensation of hunger, and reduces the appetite. In the process of treatment, needs in the food volume are reduced, and owing to this doesn't change for the worse life's quality, a person takes an enjoyment in eating and at the same time reduces his or her weight.

The percent of cellulose in some food can be from 20% to 65%, depending on the extent of a person's excess weight. The quantity of the cellulose in culinary products decreases in the percentage sense when it is necessary to slowly get thin for a person with a small excess weight, and with the purpose of the maintenance of the body's normal weight. A content in the food 60%-65% of the cellulose with addition to it mainly vegetables (except potatoes) reduces body weight every day about 1-1.5 pounds. The course of treatment continues until a normalization of body weight. And so there is a vicious circle between the taking of a meal and obesity becomes severed. It has been known for a long time that the concept “eat and then pick up flesh” is replaced with the new: “eat and get thin”. After the normalization of a person's weight, removing on usual diet doesn't attend with any difficulties, since the organism in time of the treatment didn't lose the habit of the acceptance of food.

The treatment can carry out as in-patient as in ambulatory conditions. It is expected that a large selection of food with the different substance of cellulose will be produced by the food industry and released for sale in the commercial network.

What is claimed is:

1. The invention Method of Treatment and Prophylaxis of People's Adiposity includes:
the diet with high content in the food of nontraditional cellulose of plant or animal origin as the nonmedicamental way of treatment and prophylaxis of people's adiposity and overweight;
the cellulose as an ingredient of food which increases the volume of the food's indigested part into the stomach and bowels;
the removal of the person's hungry feeling in a way of consumption of not surrendered to fermentative digestion a calorie-less mass filling the stomach and bowels;
the cellulose as a medical means ensuring a satiety by filling the stomach with a volume of mass, owing to what the organism's power requirements cover at the expense of the expenditure of a cumulative with a person excess fat, and so his or her weight reduces;
the inclusion in a food rich in the cellulose parts of plants, which before were not used in a person's meal;
the cellulose and other substances in a state of undigested in the stomach and in bowels mass, which meets the person's individual requirements in food's volume and removes the syndrome hyperorexia by people with obesity;
the cellulose coupled with calorie-less food, thanks to what is provided the normal function of digestive organs.

2. Method of Treatment and Prophylaxis of People's Adiposity includes:
all parts (stalks, leaves, bran, and others, with the exception of fruit) of eatable plants, and also wood (of the birch, fruit, and other trees) rich in the cellulose as used in the traditional meal (for example, dill's stalks, beet's leaves), as unused, but no exercising the harmful effect on the human organism; and also the cellulose of the animal origin (fascicles, tendons of muscle tissues etc.).

3. Method of Treatment and Prophylaxis of People's Adiposity includes:
the industrial manufacture of different culinary dishes, bread, confectionary, sausage, and other eatable products with an addition of nontraditional cellulose of plant or animal origin in a crushed or ground condition after cooking as the calorie-less addition increasing a food volume to reduce the person's weight;
the sale of any eatable food with special addition of the cellulose of plant or animal origin, and other substances that are indigestible in the stomach and in bowels.
